(19) United States
(12) Patent Application Publication
(10) Pub. No.: US 2010/0197809 A1
(43) Pub. Date: Aug. 5, 2010

(54) SKIN SOLUTION AND PREPARATION METHOD THEREOF

(76) Inventor: Alvaro Ernesto ORTIZ, Miami, FL (US)

Correspondence Address: CONLEY ROSE, P.C.;David A. Rose, P. O. BOX 3267, HOUSTON, TX, 77253-3267, US

(21) Appl. No.: 12/365,527

(22) Filed: Feb. 4, 2009

(51) Int. Cl. A61K 47/42; A61K 47/46

(52) U.S. Cl. 514/773; 514/783

(57) ABSTRACT

A skin solution for transporting molecules with high molecular weight to the skin, and/or moisturizing the epidermal tissues of the skin, and/or alternating the membrane permeability of the skin, and/or softening any type of callus, corns, nail folds or dry skin and facilitating the removal of the same. A method for preparing the skin solution, the solution being sprayable to form tiny droplets to be readily absorbed by the skin.

### TECHNICAL FIELD

The invention relates to a skin solution and related methods for preparing the same. With greater particularity the present invention relates to softening the skin and increasing the penetration of high molecular weight compounds at the site where the skin solution is applied.

### BACKGROUND ART

Topical therapy permits drug application to a specific disease site in high concentrations with little systemic influence. This can yield an efficient dosage which minimizes the potential of side effects. Although little quantitative data are available on permeation of specific drugs, it is known that the percutaneous absorption of a drug varies greatly dependent on numerous factors. The principal barrier affecting permeation of drugs is the stratum corneum. Moreover, many cosmetic products are in ointment base or cream base, both of them have turned out to be excellent growth mediums for bacteria which provides undesirable and possible contamination of the skin.

Consequently, there is a need for a skin solution which is physically and chemical stable and is cosmetically acceptable to transport the active chemicals or drugs into the skin at the site where the skin solution is applied. There is also a need for a sprayable skin solution forming small droplets so that compounds with molasses consistency can be readily absorbed by the skin.

### SUMMARY OF INVENTION

It is an object of the invention to provide a skin solution containing a natto extract to transport molecules with high molecular weight deep into the skin. The solution or composition will moisturize the dermis and thereby increase the penetration of said molecules at the site applied with the skin solution.

It is a further object of the invention to provide a skin solution which forms a barrier against moisture loss for a prolonged period of time after application and results in a higher liquid content retained by the cells.

It is a further object of the invention to provide a skin solution which softens and hydrates the superficial epidermal tissue if the tissue has become dry, cracked or impervious to penetration of drugs or chemicals. The composition will alter the membrane permeability of the skin and will soften any type of callus, corns, nail folds or dry skin in a few minutes and also facilitates the removal of said callus or dry skin.

It is a further object of the invention to provide a skin solution which will be physically and chemically stable and efficiently transport desired chemicals or drugs at the site on the skin applied with the skin solution.

It is a further object of the invention to provide a skin solution for treating dermal and mucosal disorders such as psoriasis, atopic dermatitis, pruritus, acne, rosacea, erithema and skin conditions associated with diabetes mellitus.

It is a further object of the invention to provide a skin solution which can kill bacterial, virus, germs or fungus on the site where the skin solution is applied.

It is a further object of the invention to provide a skin solution which is sprayable to form tiny droplets and can be readily absorbed by the skin. The delivery dosage could be measured per application.

It is a further object of the invention to provide a skin solution which is able to be a base liquid for a shampoo, conditioner, lotion or any other personal care product.

It is a further object of the invention to provide a method for preparing a stable aqueous composition containing a natto extract to transport molecules with high molecular weight deep into the skin. The composition will moisturize the dermis and thereby increase the penetration of said molecules to the site applied with the skin solution. For example, the composition should be stable for at least 6 months in a typical storage environment from 5 to 50 degrees Celsius.

It is a further object of the invention to provide a method for preparing stable aqueous composition by combining cleaning agent, ethoxylated castor oil derivatives and cationic quaternary ammonium salt to decompose the natto extract.

### BRIEF DESCRIPTION OF THE FIGURE

FIG. 1 is a flow chart of the method steps of preparing the skin solution.

### DETAILED DESCRIPTION OF INVENTION

A skin solution containing a natto extract, skin-softening agent, stabilizing agent, moisturizing agent, cleaning agent, ethoxylated castor oil derivatives and water-dispersible salt. The solution may contain an alcohol. Preferably, the skin solution transports molecules with high molecular weight deep into the dermis and increases the penetration of said molecules to the site applied with the skin solution. When the epidermal tissues become dry, cracked or impervious to penetration, the composition facilitates the action of said molecules across the skin to hydrate said epidermal tissues. The composition is able to soften the skin and to alter the membrane permeability of the skin.

In the described embodiments, disclosed are methods of preparing the skin solution containing natto extract, skin-softening agent, moisturizing agent, cationic quaternary ammonium salt, cleaning agent, ethanolamide derivatives, water-dispersible salt and alcohol.

The term “natto extract”, as used herein, is extracted from a traditional Japanese food called natto which is made from fermented soybeans by bacteria, such as Bacillus subtilis natto. The natto may be natto powder, for example, that available commercially. The natto extract is able to penetrate into the dermis and to carry the molecules with higher molecular weight across the skin barrier, to obtain the desired results. The natto contains nattokinase, prourokinase activator enzyme, fibrinolysis accelerating substances (FAS), vitamin K2, soybean, polyamine, spelmin, spelmigen daidzein, genistein, isoflavones, phytoestrogen and the chemical element selenium. The natto extract is selected from a group consisting of nattokinase, poly-g-glutamic acid, fibrinolysis accelerating substance, prourokinase activator enzyme and their mixture thereof. Alternatively, the natto extract is selected from a group consisting of fibrinolysis accelerating substance and prourokinase activator enzyme and mixtures thereof. Preferably, the concentration of the natto extract is from 0.5 to 2% by weight.

The said skin-softening agent is selected from a group consisting of one or more of natto extract, allantoin and di-isobutyl cresoxy ethoxy ethyl dimethyl benzyl ammonium chloride. Preferably, the concentration of the allantonin is present from 0.1 to 60% by weight and the concentration of di-isobutyl cresoxy ethoxy ethyl dimethyl benzyl ammonium chloride monohydrate is 0.001 to 0.5% by weight.

The stabilizing agent of the present invention is selected from a group consisting of one or more of allantoin and phenoxyethaol. The concentration of the phenoxyethanol in this specific embodiment is present from 0.001 to 0.5% by weight. The allantoin stabilizes the urea and prevents the same from decomposing in water to produce ammonia. Preferably, the concentration of the allantoin is present from 0.1 to 60% by weight. More preferably, the concentration of the allantoin is present at 0.5% by weight. The allantoin of the present invention also produces a whitening effect on the skin when applied topically.

The moisturizing agent of the present invention is selected from a group consisting of one or more of urea and natto extract. The concentration of the urea is present from 1% to 50% by weight and the concentration of the natto extract is 0.5 to 2% by weight.

The cleaning agent of the present invention is selected from a group consisting of one or more of nonylphenol ethoxylate, cocamido propyl betaine and castor oil ethoxylate. The concentration of the nonylphenol ethoxylate is from 0.001 to 2% by weight. Preferably, the concentration of the nonylphenol ethoxylate is from 0.0125 to 1% by weight, the concentration of the cocamido propyl betaina is from 0.001 to 5% by weight and the concentration of the ethoxylated castor oil is from 0.001 to 0.5% by weight.

The water-dispersible salt of the present invention is selected from a group consisting of one or more of chlorides, acetates, sulfates, nitrates, phosphates and organic salts. It is preferably a cationic quaternary ammonium salt, being selected from a group consisting of one or more of cocamido propyl betaina, di-isobutyl cresoxy ethoxy ethyl dimethyl benzyl ammonium chloride monohydrate, myristyl dimethylbenzene ammonium chloride, benzalkonium chloride, cetylpyridinium chloride, coconut dimethyl benzyl ammonium chloride, stearyl dimethyl benzyl ammonium chloride, alkyl dimethyl benzyl ammonium chloride, alkyl diethyl benzyl ammonium chloride, alkyl dimethyl benzyl ammonium bromide, di-isobutyl phenoxy ethoxy ethyl trimethyl ammonium chloride, di-isobutyl phenoxy ethoxy ethyl dimethyl alkyl ammonium chloride, methyl-dodecylbenzyl trimethyl ammonium chloride, cetyl trimethyl ammonium bromide, octadecyl dimethyl ethyl ammonium bromide, cetyl dimethyl ethyl ammonium bromide, octadecenyl-9-dimethyl ethyl ammonium bromide, dioctyl dimethyl ammonium chloride, dodecyl trimethyl ammonium chloride, octadecyl trimethyl ammonium chloride, octadecyl trimethyl ammonium bromide, hexadecynyl trimethyl ammonium iodine and octyltrimethyl ammonium fluoride. Preferably, the cationic quaternary ammonium salts are di-isobutyl cresoxy ethoxy ethyl dimethyl benzyl ammonium chloride monohydrate and cocamido propyl betaina; and the concentration of the cocamido propyl betaina is present from 0.001 to 5% by weight and the concentration of the di-isobutyl cresoxy ethoxy ethyl dimethyl benzyl ammonium chloride monohydrate is present from 0.001 to 0.5% by weight. More preferably, the concentration of the di-isobutyl cresoxy ethoxy ethyl dimethyl benzyl ammonium chloride monohydrate is present from 0.0025 to 0.2% by weight. Most preferably, the concentration of the di-isobutyl cresoxy ethoxy ethyl dimethyl benzyl ammonium chloride monohydrate is 0.15% by weight.

The quaternary ammonium salt is used to break the surface tension and permit the improved penetration of the natto extract into the site applied with the skin solution. There is a synergy when the cationic quaternary ammonium salt is used in combination with nonylphenol ethoxylate. The addition of nonylphenol ethoxylate provides a synergistic effect of greater penetration of the natto extract into the skin.

The ethoxylated castor oil derivative of the present invention are selected from a group consisting one or more of ethoxylated castor oil and undecylenamide DEA. The concentration of the undecylenamide DEA is present from 0.001 to 0.5% by weight and the concentration of the ethoxylated castor oil is from 0.001 to 0.5% by weight. Preferably, the concentration of ethoxylated castor oil is from 0.0125 to 1% by weight.

The skin solution of the present invention can also contain additives, preservatives, plant extracts, vitamins and their combinations thereof.

Examples of additives are azelaic acid, cozyme CQ10, biazulene, lecithin, ellagic acid, shea butter, dioxybenzone, avobenzone, zinc oxide, hyaluronic acid and their combinations thereof.

Examples of preservatives are methyl paraben, propyl paraben and their combinations thereof.

Examples of plant extract are tea tree extracts, pommergranate extract, aloe vera extract and their combinations thereof.

Examples of vitamins are vitamin a, vitamin c, vitamin e and their combinations thereof.

An example of alcohol is ethanol. The concentration of ethanol is 5% by volume.

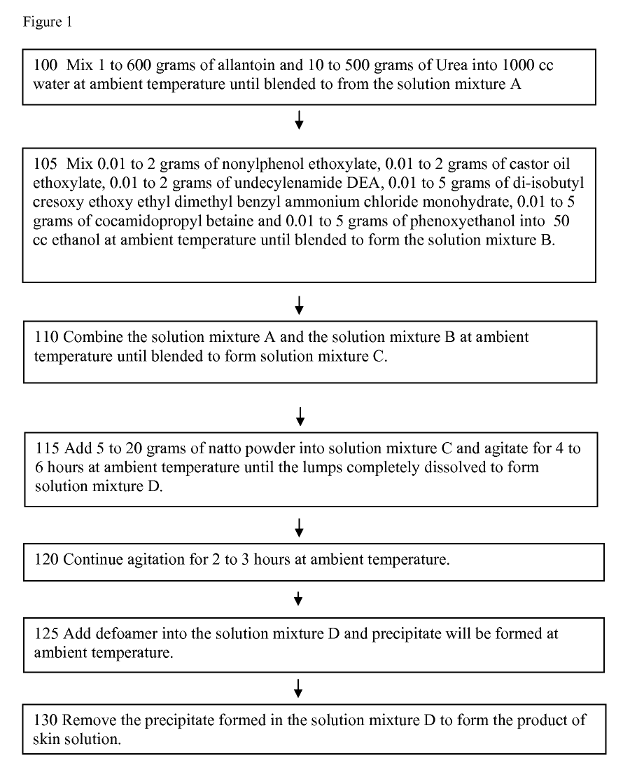

The skin solution is in the form of a solution. Preferably, the composition is sprayable to form tiny droplets to be readily absorbed by the skin where the solution is applied. FIG. 1 is the flow chart of preparing the skin solution (10). The skin solution is prepared and mixed at ambient temperature. An amount of 1 to 600 grams of allantoin and 10 to 500 grams of urea is mixed with 1000 cc distilled water at ambient temperature until blended to form solution mixture A (100). An amount of 0.01 to 2 grams of nonylphenol ethoxylate, 0.01 to 2 grams of castor oil ethoxylate, 0.01 to 2 grams of undecylenamide DEA, 0.01 to 5 grams of di-isobutyl cresoxy ethoxy ethyl dimethyl benzyl ammonium chloride monohydrate, 0.01 to 5 grams of cocamidopropyl betaine and 0.01 to 5 grams of phenoxyethanol is mixed with 50 cc ethanol in a separate container at ambient temperature until blended to form the solution mixture B (105). The solution mixture B is combined with solution mixture A at ambient temperature until blended to form solution mixture C (110). An amount of 5 to 20 grams of natto powder is added into the solution mixture C and agitated for 4 to 6 hours at ambient temperature until the lumps completely dissolved to form solution mixture D (115). The solution mixture D is continuously agitated for 2 to 3 hours at ambient temperature (120) and followed by adding the defoamer into the solution mixture D and precipitate will be formed at ambient temperature. The end point of the reaction is reached when the pH reaches about 6.5. The precipitate is removed to form the product of skin solution (130).

In another embodiment, there is provided a topically applicable trans-dermal active component transport solution, comprising urea, urea stabilizing agent, natto extract, one or more ethoxylate entities and a cationic quaternary ammonium salt. Save where defined in this paragraph, the above text in relation to the skin solution is equally applicable to the transport solution. The ethoxylate entity is one or more of nonyl phenol ethoxylate, castor oil ethoxylate and undecylenamide DEA. The natto extract is selected from a group consisting of fibrinolysis accelerating substance, prourokinase activator enzyme and mixtures thereof. The transport solution may further comprise ethanol and cocamidopropyl betaina.

### INDUSTRIAL APPLICABILITY

The present invention relates to a skin solution for transporting molecules with high molecular weight to the skin. More particularly, the present invention relates to a skin solution containing a natto extract and a method to prepare the same.

1. A skin solution comprising natto extract, skin-softening agent, stabilizing agent, moisturizing agent, cleaning agent, ethoxylated castor oil derivative and water-dispersible salt.

2. The skin solution of claim 1 wherein the natto extract is selected from a group consisting of fibrinolysis accelerating substance, prourokinase activator enzyme and mixtures thereof.

3. The skin solution of claim 1, where the natto extract is selected from the group consisting of nattokinase, poly-g-glutamic acid, fibrinolysis accelerating substance, prourokinase activator enzyme and mixtures thereof,

4. The skin solution of claim 1 wherein said skin-softening agent is selected from a group consisting of one or more of natto extract, allantoin and di-isobutyl cresoxy ethoxy ethyl dimethyl benzyl ammonium chloride.

5. The skin solution of claim 1 wherein said stabilizing agent is selected from a group of consisting of one or more of allantoin and phenoxyethanol.

6. The skin solution of claim 1 wherein said moisturizing agent is selected from a group consisting of one or more of urea and natto extract.

7. The skin solution of claim 1 wherein the water dispersible salt is a cationic quaternary ammonium salt which is selected from a group consisting of one or more of cocamido propyl betaina, di-isobutyl cresoxy ethoxy ethyl dimethyl benzyl ammonium chloride monohydrate, di-isobutyl phenoxy ethoxy ethyl dimethyl benzyl ammonium chloride, myristyl dimethylbenzene ammonium chloride, benzalkonium chloride, cetyl pyridinium chloride, coconut dimethyl benzyl ammonium chloride, stearyl dimethyl benzyl ammonium chloride, alkyl dimethyl benzyl ammonium chloride, alkyl diethyl benzyl ammonium chloride, alkyl dimethyl benzyl ammonium bromide, di-isobutyl phenoxy ethoxy ethyl trimethyl ammonium chloride, di-isobutyl phenoxy ethoxy ethyl dimethyl alkyl ammonium chloride, methyl-dodecylbenzyl trimethyl ammonium chloride, cetyl trimethyl ammonium bromide, octadecyl dimethyl ethyl ammonium bromide, cetyl dimethyl ethyl ammonium bromide, octadecenyl-9-dimethyl ethyl ammonium bromide, dioctyl dimethyl ammonium chloride, dodecyl trimethyl ammonium chloride, octadecyl trimethyl ammonium chloride, octadecyl trimethyl ammonium bromide, hexadecynyl trimethyl ammonium iodine and octyltrimethyl ammonium fluoride.

8. The skin solution of claim 1 wherein said cleaning agent is selected from a group consisting of one or more of nonylphenol ethoxylate, cocamido propyl betaine and castor oil ethoxylate.

9. The skin solution of claim 1 wherein said ethoxylated castor oil derivate is selected from a group consisting one or more of ethoxylated castor oil and undecylenamide DEA.

10. The skin solution of claim 2, wherein the amount of said natto extract is 0.5 to 2% by weight.

11. The skin solution of claim 5 wherein the amount of said allantoin is 0.1 to 60% by weight.

12. The skin solution of claim 5 wherein the amount of said phenoxyethanol is 0.001 to 0.5% by weight.

13. The skin solution of claim 6 wherein the amount of said urea is 1 to 50% by weight.

14. The skin solution of claim 7 wherein the amount of said cocamido propyl betaina is 0.001 to 5% by weight.

15. The skin solution of claim 8 wherein the amount of said nonylphenol ethoxylate is 0.001 to 2% by weight.

16. The skin solution of claim 9 wherein said the amount of each ethoxylated castor oil derivate is 0.001 to 2% by weight.

17. A topically applicable trans-dermal active component transport solution, comprising urea, urea stabilizing agent, natto extract, one or more ethoxylate entities and a cationic quaternary ammonium salt.

18. The solution of claim 17, wherein the ethoxylate entity is one or more of nonyl phenol ethoxylate, castor oil ethoxylate and undecylenamide DEA.

19. The solution of claim 18, wherein the natto extract is selected from a group consisting of fibrinolysis accelerating substance, prourokinase activator enzyme and mixtures thereof.

20. The solution of claim 19 wherein the vehicle further comprises ethanol and cocamidopropyl betaina.
